(19) United States
(12) Patent Application Publication
(10) Pub. No.: US 2019/0307428 A1
(43) Pub. Date: Oct. 10, 2019

(54) METHODS AND APPARATUS FOR CONFIGURING AN ULTRASOUND SYSTEM WITH IMAGING PARAMETER VALUES

(71) Applicants: Nathan Silberman, Brooklyn, NY (US); Alex Rothberg, New York, NY (US); Israel Malkin, New York, NY (US); Karl Thiele, Andover, MA (US); Tyler S. Ralston, Clinton, CT (US); Christophe Meyer, New York, NY (US)

(72) Inventors: Nathan Silberman, Brooklyn, NY (US); Alex Rothberg, New York, NY (US); Israel Malkin, New York, NY (US); Karl Thiele, Andover, MA (US); Tyler S. Ralston, Clinton, CT (US); Christophe Meyer, New York, NY (US)

(73) Assignee: Butterfly Network, Inc., Guilford, CT (US)

(21) Appl. No.: 16/379,498

(22) Filed: Apr. 9, 2019

Related U.S. Application Data
(60) Provisional application No. 62/655,162, filed on Apr. 9, 2018.

(51) Int. Cl. A61B 8/00; A61B 8/08

(52) U.S. Cl. CPC A61B 8/54; A61B 8/467; A61B 8/5269; A61B 8/5207

(57) ABSTRACT

Aspects of the technology described herein relate to configuring an ultrasound system with imaging parameter values. In particular, certain aspects relate to configuring an ultrasound system to produce a plurality of sets of ultrasound images, each respective set of the plurality of sets of ultrasound images being produced with a different respective set of a plurality of sets of imaging parameter values; obtaining, from the ultrasound system, the plurality of sets of ultrasound images; determining a set of ultrasound images from among the plurality of sets of ultrasound images that has a highest quality; and based on determining the set of ultrasound images from among the plurality of sets of ultrasound images that has the highest quality, automatically configuring the ultrasound system to produce ultrasound images using a set of imaging parameter values with which the set of ultrasound images that has the highest quality was produced.

### CROSS-REFERENCE TO RELATED APPLICATIONS

This application claims the benefit under 35 U.S.C. § 119(e) of U.S. Patent Application Ser. No. 62/655,162, filed Apr. 9, 2018 under Attorney Docket No. B1348.70077US00, and entitled “METHODS AND APPARATUS FOR CONFIGURING AN ULTRASOUND SYSTEM WITH IMAGING PARAMETER VALUES,” which is hereby incorporated herein by reference in its entirety.

### FIELD

Generally, the aspects of the technology described herein relate to ultrasound data collection. Some aspects relate to configuring an ultrasound system with imaging parameter values.

### BACKGROUND

Ultrasound systems may be used to perform diagnostic imaging and/or treatment, using sound waves with frequencies that are higher with respect to those audible to humans. Ultrasound imaging may be used to see internal soft tissue body structures, for example to find a source of disease or to exclude any pathology. When pulses of ultrasound are transmitted into tissue (e.g., by using a pulser in an ultrasound imaging device), sound waves are reflected off the tissue, with different tissues reflecting varying degrees of sound. These reflected sound waves may then be recorded and displayed as an ultrasound image to the operator. The strength (amplitude) of the sound signal and the time it takes for the wave to travel through the body provide information used to produce the ultrasound image. Many different types of images can be formed using ultrasound systems, including real-time images. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect, a method of operating an ultrasound device includes automatically imaging an anatomical target multiple times with different sets of imaging parameters; and automatically selecting for continued imaging of the anatomical target, from the different sets of imaging parameters, a first set of imaging parameters. In some embodiments, the first set of imaging parameters represents those imaging parameters determined to produce images of the anatomical target of a highest quality from among the sets of imaging parameters

According to another aspect, a method includes configuring, with a processing device, an ultrasound system to produce a plurality of sets of ultrasound images, each respective set of the plurality of sets of ultrasound images being produced with a different respective set of a plurality of sets of imaging parameter values; obtaining, from the ultrasound system, the plurality of sets of ultrasound images; determining a set of ultrasound images from among the plurality of sets of ultrasound images that has a highest quality; and based on determining the set of ultrasound images from among the plurality of sets of ultrasound images that has the highest quality, automatically configuring the ultrasound system to produce ultrasound images using a set of imaging parameter values with which the set of ultrasound images that has the highest quality was produced.

In some embodiments, configuring the ultrasound imaging device to produce the plurality of sets of ultrasound images is performed based on detecting that the ultrasound system has begun imaging a subject after not imaging the subject for a threshold period of time. In some embodiments, detecting that the ultrasound system has begun imaging the subject after not imaging the subject for the threshold period of time includes configuring the ultrasound system with a low-power set of imaging parameter values that uses less power than the plurality of sets of imaging parameter values.

In some embodiments, determining the set of ultrasound images from among the plurality of sets of ultrasound images that has the highest quality includes determining the set of ultrasound images from among the plurality of sets of ultrasound images for which a view classifier has a highest confidence that the view classifier recognizes an anatomical region in the set of ultrasound images. In some embodiments, determining the set of ultrasound images from among the plurality of sets of ultrasound images that has the highest quality includes calculating an image sharpness metric for each of the plurality of sets of ultrasound images. In some embodiments, determining the set of ultrasound images from among the plurality of sets of ultrasound images that has the highest quality includes calculating a pixel variation metric for each of the plurality of sets of ultrasound images. In some embodiments, determining the set of ultrasound images from among the plurality of sets of ultrasound images that has the highest quality includes calculating a noise metric for each of the plurality of sets of ultrasound images. In some embodiments, determining the set of ultrasound images from among the plurality of sets of ultrasound images that has the highest quality includes calculating a total variation metric for each of the plurality of sets of ultrasound images. In some embodiments, determining the set of ultrasound images from among the plurality of sets of ultrasound images that has the highest quality includes calculating a pixel intensity metric for each of the plurality of sets of ultrasound images.

In some embodiments, the method further includes generating an instruction for a user to hold substantially stationary an ultrasound imaging device configured for operative communication with the processing device while the ultrasound system is producing the plurality of sets of ultrasound images. In some embodiments, the method further includes generating a notification for a user that indicates the set of imaging parameter values with which the set of ultrasound images that has the highest quality metric was produced.

In some embodiments, the plurality of sets of imaging parameter values include ultrasound imaging presets each optimized for imaging a particular anatomical region among a plurality of anatomical regions. In some embodiments, the plurality of anatomical regions include a plurality of anatomical regions typically imaged during a particular ultrasound imaging protocol. In some embodiments, the method further includes receiving an input from a user that the user will be performing the particular ultrasound imaging protocol. In some embodiments, the plurality of sets of imaging parameter values include preferred sets of imaging parameter values associated with a user.

In some embodiments, configuring the ultrasound system to produce the plurality of sets of ultrasound images includes configuring the ultrasound system to: transmit a plurality of sets of ultrasound waves into a subject using the plurality of sets of imaging parameter values, wherein the plurality of sets of imaging parameter values relate to ultrasound transmission; and generate each of the plurality of sets of ultrasound images from a different set of reflected ultrasound waves each corresponding to one of the plurality of sets of transmitted ultrasound waves. In some embodiments, configuring the ultrasound system to produce the plurality of sets of ultrasound images includes configuring the ultrasound system to: transmit a single set of ultrasound waves into a subject; and generate each of the plurality of sets of ultrasound images from a single set of reflected ultrasound waves corresponding to the single set of transmitted ultrasound waves using the plurality of sets of imaging parameter values, wherein the plurality of sets of imaging parameter values relate to ultrasound image generation. In some embodiments, the ultrasound system includes the processing device and an ultrasound imaging device. In some embodiments, the ultrasound system includes the processing device.

According to another aspect, a method includes transmitting one or more instructions to an ultrasound imaging device to trigger configuration of the ultrasound imaging device to produce ultrasound data using low-frequency ultrasound waves; determining whether the ultrasound data includes ultrasound waves from depths beyond a threshold depth having an amplitude that exceeds a threshold amplitude value; and based on determining whether the ultrasound data includes ultrasound waves from depths beyond a threshold depth having an amplitude that exceeds a threshold amplitude value, determining whether to transmit one or more instructions to the ultrasound imaging device to trigger automatic configuration of the ultrasound imaging device to produce ultrasound data using low-frequency ultrasound waves or high-frequency ultrasound waves.

In some embodiments, transmitting the one or more instructions to the ultrasound imaging device to trigger configuration of the ultrasound imaging device to produce ultrasound data using low-frequency ultrasound waves is performed based on detecting that the ultrasound system has begun imaging a subject after not imaging the subject for a threshold period of time. In some embodiments, detecting that the ultrasound system has begun imaging the subject after not imaging the subject for the threshold period of time includes configuring the ultrasound system with a low-power set of imaging parameter values that uses less power than the plurality of sets of imaging parameter values. In some embodiments, the amplitude of the ultrasound waves includes the amplitude of the ultrasound waves received at the ultrasound system after a time required for the ultrasound waves to travel from the ultrasound system to the threshold depth and reflect back from the threshold depth to the ultrasound system. In some embodiments, determining whether the ultrasound data includes ultrasound waves from depths beyond the threshold depth having the amplitude that exceeds the threshold amplitude value includes inputting the ultrasound data to a neural network trained to determine whether the inputted ultrasound data includes the ultrasound waves from depths beyond the threshold depth having the amplitude that exceeds the threshold amplitude value. In some embodiments, the threshold depth includes a depth between approximately 5-20 cm. In some embodiments, the low-frequency ultrasound waves include ultrasound waves having a frequency between approximately 1-5 MHz. In some embodiments, the high-frequency ultrasound waves include ultrasound waves having a frequency between approximately 5-12 MHz.

Some aspects include at least one non-transitory computer-readable storage medium storing processor-executable instructions that, when executed by at least one processor, cause the at least one processor to perform the above aspects and embodiments. Some aspects include an ultrasound system having a processing device configured to perform the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 shows an example process for configuring an ultrasound imaging device with imaging parameter values in accordance with certain embodiments described herein;

FIG. 2 shows an example graphical user interface (GUI) generated by a processing device that may be in operative communication with an ultrasound imaging device, in which the GUI shows a notification to hold the ultrasound imaging device stationary;

FIG. 3 shows an example GUI generated by the processing device, in which the GUI shows a textual notification of an automatically selected preset;

FIG. 4 shows an example GUI generated by the processing device, in which the GUI shows a pictorial notification of an automatically selected preset.;

FIG. 5 shows a non-limiting alternative to the pictorial notification of FIG. 4;

FIG. 6 shows another non-limiting alternative to the pictorial notifications of FIGS. 4 and 5;

FIG. 7 shows an example process for configuring an ultrasound system with imaging parameter values in accordance with certain embodiments described herein;

FIG. 8 shows a schematic block diagram illustrating aspects of an example ultrasound system upon which various aspects of the technology described herein may be practiced;

FIG. 9 is a schematic block diagram illustrating aspects of another example ultrasound system upon which various aspects of the technology described herein may be practiced; and

FIG. 10 shows an example convolutional neural network that is configured to analyze an image.

### DETAILED DESCRIPTION

An ultrasound system typically includes preprogrammed parameter values for configuring the ultrasound system to image various anatomical features. For example, a given anatomical feature may be located at a certain depth from the surface of a subject, and the depth may determine imaging parameters such as frequency. Thus, for example, a user wishing to scan a subject's heart may manually select imaging parameter values associated with the heart on the ultrasound imaging system, and this selection may configure the ultrasound system with the preprogrammed parameter values for cardiac ultrasound imaging. The user may, for example, make the selection by choosing a menu option on a display screen or pressing a physical button.

The inventors have recognized that in some embodiments, the ease for a user to perform ultrasound imaging may be improved by automatically determining imaging parameter values for imaging a particular region of a subject. In particular, the inventors have recognized that multiple sets of imaging parameter values may be tested to determine which set is most appropriate for imaging a particular region on a subject. Testing the multiple sets of imaging parameter values may include obtaining, from the ultrasound system, multiple sets of ultrasound images produced from the same location on a subject using different sets of imaging parameter values. Once sets of ultrasound images have been produced using all the sets of imaging parameter values to be tested, which of the imaging parameters values produced the “best” set of ultrasound images may be determined by calculating a quality for each of the sets of ultrasound images. The quality may be calculated, for example, as a confidence that a view classifier recognizes an anatomical region in the set of ultrasound images. The ultrasound system may then be configured to continue imaging with the imaging parameter values that produced the “best” set of ultrasound images. Accordingly, the user may not need to manually select the imaging parameter values for the imaging the region of interest.

For example, a user may place an ultrasound imaging device included in the ultrasound system at a single location at the subject's heart. The ultrasound system may automatically produce multiple sets of ultrasound images from that one location at the subject's heart using imaging parameter values optimized for the heart, the abdomen, the bladder, or other anatomical features or regions. The ultrasound system may then determine that the imaging parameter values for the heart produced the “best” data, and configure itself to continue imaging using the imaging parameter values for the heart. The user may then continue to produce, using the ultrasound system configured with the imaging parameter values for the heart, ultrasound images from different locations at the heart and with different orientations of the ultrasound imaging device relative to the heart.

The inventors have further recognized that in some embodiments, a single test, namely production of ultrasound data from a region of interest on a subject using low-frequency ultrasound waves, may be used to determine whether low-frequency ultrasound waves or high-frequency ultrasound waves are appropriate for use in imaging the region of interest. Certain anatomical structures are located shallow within human subjects (e.g., 4-10 cm below the skin) and certain anatomical structures are located deep within human subjects (e.g., 10-25 cm below the skin). High-frequency ultrasound waves may be used to produce ultrasound images having higher axial resolution than images produced using low-frequency ultrasound waves. However, high-frequency ultrasound waves may be attenuated within a subject over a given distance than low-frequency ultrasound waves. Therefore, high-frequency ultrasound waves may be appropriate for ultrasound imaging of shallow anatomical structures, and low-frequency ultrasound may be appropriate for ultrasound imaging of deep anatomical structures. To determine whether low-frequency ultrasound waves are appropriate for use in imaging the region of interest, the processing circuitry may determine whether substantial echoes are reflected back from beyond a threshold depth following transmission of test low-frequency ultrasound waves. If substantial echoes are reflected back from beyond the threshold depth following transmission of the test low-frequency ultrasound waves, this may indicate that deep anatomical structures are present and low-frequency ultrasound waves are appropriate for use. If substantial echoes are not reflected back from beyond the threshold depth following transmission of the test low-frequency ultrasound waves, this may indicate that deep anatomical structures are not present and high-frequency ultrasound waves are appropriate for use. This may be considered a method for automatically configuring an ultrasound system for deep or shallow ultrasound imaging.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

As referred to herein, producing a set of ultrasound images should be understood to mean transmitting ultrasound waves, receiving reflected ultrasound waves, and generating a set of ultrasound images from the reflected ultrasound waves. A set of ultrasound images may include one or more ultrasound images. As referred to herein, producing a set of ultrasound images with a set of imaging parameter values should be understood to mean producing the set of ultrasound images using an ultrasound system that has been configured with the set of imaging parameter values.

As referred to herein, producing a set of ultrasound images using low-frequency waves should be understood to mean transmitting low-frequency ultrasound waves, receiving reflected ultrasound waves, and generating a set of ultrasound images from the reflected ultrasound waves. Similarly, as referred to herein, producing a set of ultrasound images using high-frequency waves should be understood to mean transmitting high-frequency ultrasound waves, receiving reflected ultrasound waves, and generating a set of ultrasound images from the reflected ultrasound waves.

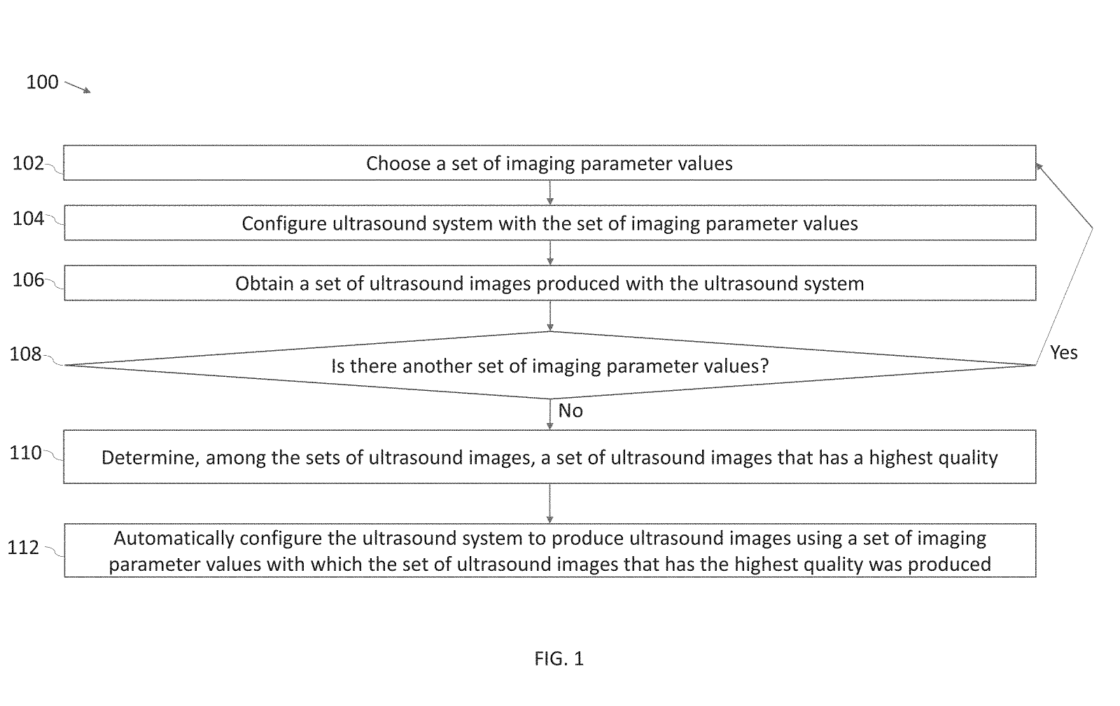

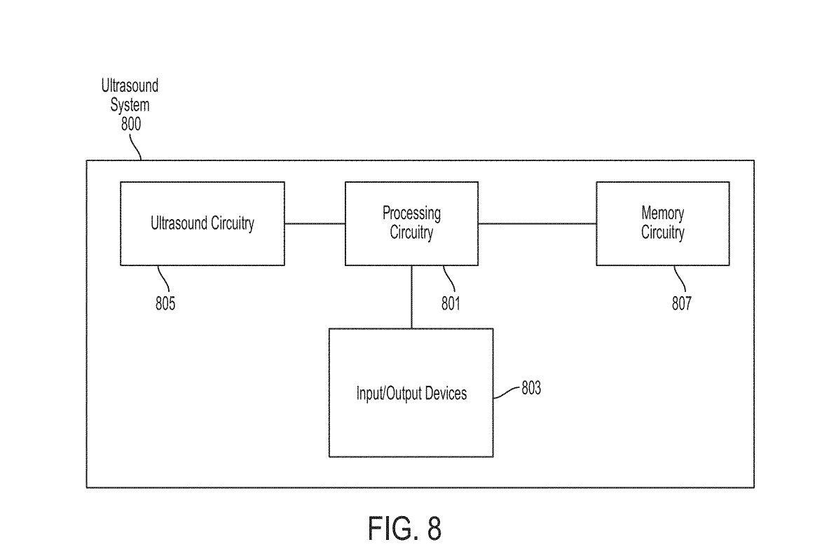

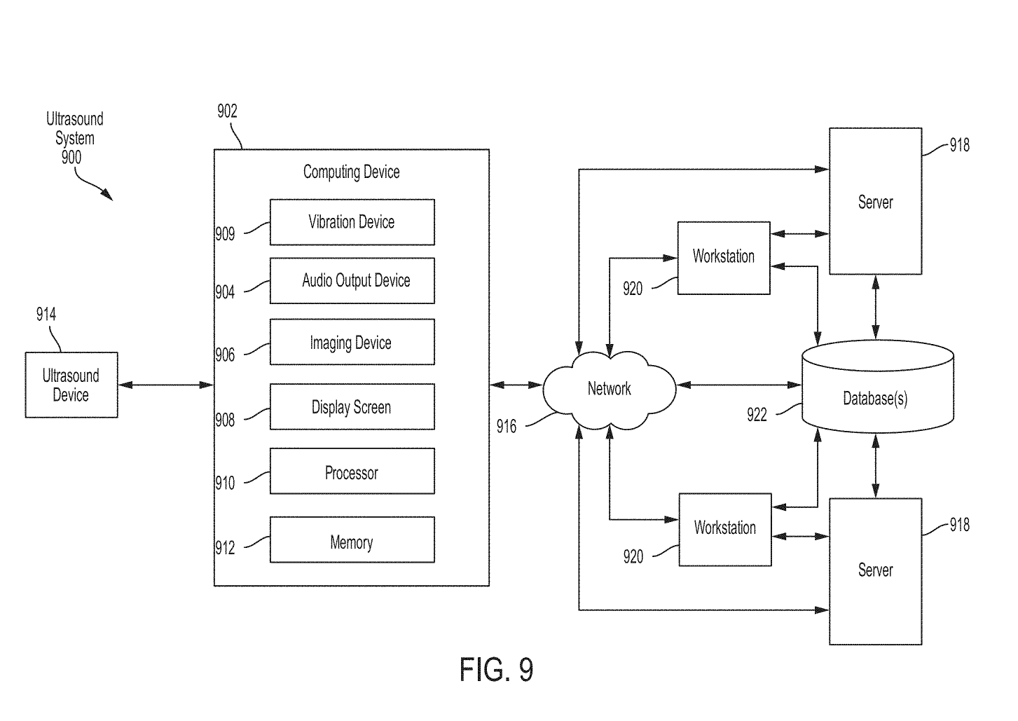

FIG. 1 shows an example process 100 for configuring an ultrasound system with imaging parameter values in accordance with certain embodiments described herein. The process 100 may be performed by, for example, processing circuitry in the ultrasound system. The ultrasound system may include an ultrasound imaging device used for imaging a subject as well as one or more external devices (e.g., a mobile phone, tablet, laptop, or server) in operative communication with the ultrasound imaging device, and the processing circuitry may be in either or both of these devices. Ultrasound systems and devices are described in more detail with reference to FIGS. 8-9.

Process 100 generally includes searching through and testing multiple sets of imaging parameter values to select, based on certain criteria, a set that is most appropriate for imaging a particular region on a subject. Testing the multiple sets of imaging parameter values includes obtaining, from the ultrasound system, multiple sets of ultrasound images produced from the same location on a subject using different sets of imaging parameter values (acts 102, 104, 106, and 108). In particular, during each iteration through acts 102, 104, and 106, a different set of ultrasound images is produced using a different set of imaging parameter values. Once sets of ultrasound images have been produced using all the sets of imaging parameter values to be tested, process 100 determines which of the imaging parameters values produced the “best” set of ultrasound images, as determined by calculating a quality for each of the sets of ultrasound images (act 110). Process 100 further includes configuring the ultrasound system to continue imaging with the imaging parameter values that produced the “best” set of ultrasound images (act 112). For example, a user may place an ultrasound imaging device included in the ultrasound system at a single location at the subject's heart. The ultrasound system may produce multiple sets of ultrasound images from that one location at the subject's heart using imaging parameter values optimized for the heart, the abdomen, the bladder, etc. The processing circuitry may then determine that the imaging parameter values for the heart produced the “best” data, and configure the ultrasound system to continue imaging using the imaging parameter values for the heart. The user may then continue to produce, using the ultrasound system configured with the imaging parameter values for the heart, ultrasound images from different locations at the heart and with different orientations of the ultrasound imaging device relative to the heart. Accordingly, the user may not need to manually select the imaging parameter values for the heart prior to commencing imaging of the heart.

In act 102, the processing circuitry may choose values for a set of imaging parameters. The imaging parameters may be parameters governing how the ultrasound system performs ultrasound imaging. Non-limiting examples of imaging parameters that may be included in the set of imaging parameters are frequency, gain, frame rate, power, the speed of sound, and azimuthal/elevational focus.

In some embodiments, the processing circuitry may choose imaging parameter values corresponding to an ultrasound imaging preset. The ultrasound imaging preset may be a predetermined set of imaging parameter values optimized for imaging a particular anatomical region (e.g., cardiac, carotid, abdomen, extremities, bladder, musculoskeletal, uterus, as non-limiting examples). Presets may be further optimized based on the subject (e.g., a pediatric cardiac preset and an adult cardiac preset) and/or based on whether deep or superficial portions of the anatomical region are to be imaged (e.g., a musculoskeletal superficial preset and a musculoskeletal deep preset).

The ultrasound system may be programmed with a group of ultrasound imaging presets corresponding to anatomical regions that the ultrasound imaging device is capable of imaging. Each time the processing circuitry chooses a set of imaging parameter values (as described below, the ultrasound imaging device may iterate through act 102 multiple times), the processing circuitry may retrieve a different preset from the group. In some embodiments, a particular group of preferred ultrasound imaging presets may be associated with a user. For example, a user may choose preferred presets that s/he anticipates using frequently (e.g., if the user is a cardiologist, the user may choose cardiac and carotid presets). As another example, preferred presets may be associated with a user based on the user's past history (e.g., if the user most often uses cardiac and abdominal presets, the cardiac and abdominal presets may be automatically associated with the user). In such embodiments, each time the processing circuitry chooses a set of imaging parameter values, the processing circuitry may retrieve a different preset from the preferred group of presets associated with the user. In some embodiments, a user may input (e.g., by selecting an option from a menu on a graphical user interface, pressing a physical button, using a voice command) a particular ultrasound imaging protocol into the ultrasound system. The ultrasound imaging protocol may require scanning particular anatomical regions, but the order in which the user will scan the particular anatomical regions may not be known. In such embodiments, each time the processing circuitry chooses a set of imaging parameter values, the processing circuitry may retrieve a different preset from a group of presets associated with the anatomical regions that are scanned as part of the ultrasound imaging protocol. For example, a FAST (Fast Assessment with Sonography in Trauma) exam may include scanning the heart and abdomen, and therefore if the user inputs that s/he is performing a FAST exam, each time the processing circuitry chooses a set of imaging parameter values, the ultrasound system may retrieve either a cardiac preset or an abdominal preset. Another example protocol may by the Rapid Ultrasound for Shock and Hypotension (RUSH) exam, which may include collecting various views of the heart, vena cava, Morison's pouch, spleen, kidney, bladder, aorta, and lungs.

In some embodiments, each time the processing circuitry chooses a set of imaging parameter values, the processing circuitry may choose a different value from a portion of all possible values for the imaging parameters, such that after multiple iterations through act 102, the processing circuitry may have iterated through a portion of all combinations of the imaging parameters. For example, in a non-limiting illustrative example in which the only imaging parameter is frequency, if the ultrasound imaging device is capable of imaging at frequencies of 1-15 MHz, the processing circuitry may choose a different one of 1 MHz, 2 MHz, 3 MHz, 4 MHz, 5 MHz, 6 MHz, 7 MHz, 8 MHz, 9 MHz, 10 MHz, 11 MHz, 12 MHz, 13 MHz, 14 MHz, and 15 MHz during each iteration through act 102. In examples in which the processing circuitry chooses values for multiple imaging parameters (e.g., two or more of frequency, gain, frame rate, and power), the processing circuitry may choose a different combination of the imaging parameters during each iteration through act 102. In other words, the processing circuitry may iterate through a portion of the entire imaging parameter space after multiple iterations through act 102. In general, regardless of how the particular imaging parameter values are chosen, the set of imaging parameter values chosen at act 102 may be different than any other set of imaging parameter values chosen at previous iterations through act 102. The process 100 may then continue to act 104.

In act 104, the processing circuitry may configure the ultrasound system with the set of imaging parameter values chosen in act 102. In some embodiments, the processing circuitry may configure the ultrasound system with values for imaging parameters related to ultrasound transmission (e.g., the frequency of ultrasound waves transmitted by the ultrasound system into a subject). In some embodiments, the processing circuitry may configure the ultrasound system with values for imaging parameters related to ultrasound image generation (e.g., the speed of sound within the portion of the subject being imaged, azimuthal/elevational focus, etc.). In some embodiments, a processing device in operative communication with the ultrasound imaging device may transmit an instruction/instructions to the ultrasound imaging device to trigger configuration of the ultrasound imaging device with the imaging parameter values chosen in act 102. This may be helpful when the ultrasound imaging device must be configured with an image parameter value related to transmission of ultrasound waves from the ultrasound imaging device. The process 100 may then proceed to act 106.

In act 106, the processing circuitry may obtain a set of ultrasound images produced by the ultrasound system. The set of ultrasound images may be images produced with the ultrasound system as configured (in act 104) with the imaging parameter values chosen in act 102 and may be obtained from the same region of interest on the subject as data produced during a previous iteration through act 106. In embodiments in which the imaging parameters relate to ultrasound transmission, the set of ultrasound images may be produced by transmitting ultrasound waves corresponding to the set of imaging parameter values into the subject and generating the set of ultrasound images from the reflected ultrasound waves. In embodiments in which the imaging parameters relate to image generation, the set of ultrasound images may be produced from reflected ultrasound waves by using the image generation parameter values. In some embodiments, after an ultrasound imaging device has received a set of ultrasound data, the ultrasound imaging device may transmit the set of ultrasound data to a processing device in operative communication with the ultrasound imaging device, and the processing device may generate a set of ultrasound images from the set of ultrasound data. Transmission may occur over a wired communication link (e.g., over Ethernet, a Universal Serial Bus (USB) cable or a Lightning cable) or over a wireless communication link (e.g., over a BLUETOOTH, WiFi, or ZIGBEE wireless communication link). The process 100 may then proceed to act 108.

In act 108, the processing circuitry may determine if there is another set of imaging parameter values to test. If there is another set of imaging parameter values to test, the process 100 may proceed to act 102, in which another set of imaging parameter values will be chosen (act 102). Following act 102, the new set of imaging parameter values will be used to configure the ultrasound system (act 104) for producing a set of ultrasound images (act 106). If there is not another set of imaging parameter values to test, the process 100 may proceed to act 110.

Accordingly, with each iteration through acts 102, 104, and 106, a different set of ultrasound images may be obtained using a different set of imaging parameter values, producing multiple sets of ultrasound images after multiple iterations through acts 102, 104, and 106. In embodiments in which different sets of imaging parameter values related to ultrasound transmission are used, the different sets of ultrasound images may be produced by transmitting different ultrasound waves (e.g., having different frequencies) into the subject and generating different images for each set of reflected ultrasound waves. In embodiments in which different sets of imaging parameters values related to image generation are used, the different sets of ultrasound images may be produced by using different image generation parameter values to produce different ultrasound images from the same set of ultrasound waves reflected after transmitting the same set of ultrasound waves. The multiple sets of ultrasound images may be considered test data for testing which set of imaging parameter values should be used to configure the ultrasound system for continued imaging. As will be described below with reference to act 110, this testing is performed by determining, from among all the sets of ultrasound images produced during multiple iterations through acts 102, 104, and 106, which set of ultrasound images has the highest quality.

In some embodiments, the set of imaging parameters tested may include the frequency of transmitted ultrasound waves. Because the frequency of transmitted ultrasound waves may determine how well anatomical structures at a particular depth can be imaged, determining what frequency produces ultrasound images having the highest quality may help to improve the quality of imaging of anatomical structures at the region of interest.

In some embodiments, the set of imaging parameters tested may include the speed of sound within the subject. Because the speed of sound within a subject may vary depending on how much fat is at the region of interest and the types of organs/tissues at the region of interest, and because the speed of sound affects generation of ultrasound images from reflected ultrasound waves, determining what speed of sound value produces ultrasound images having the highest quality may help to improve the quality of imaging of particular individuals (e.g., those that have more fat and those that have less fat) or particular anatomical structures at the region of interest.

In some embodiments, the set of imaging parameters tested may include the azimuthal and/or elevational focus. Because the azimuthal and/or elevational focus may determine what anatomical structures are in focus in a generated image, determining what azimuthal/elevational focus produces ultrasound images having the highest quality may help to improve the quality of imaging of particular anatomical structures at the region of interest.

In act 110, the processing circuitry may determine among the sets of ultrasound images produced from iterations through acts 102, 104, and 106, a set of ultrasound images that has a highest quality. For example, the processing circuitry may calculate a value for the quality of each particular set of ultrasound images, and associate the quality value with the particular set of imaging parameter values used to produce the particular set of ultrasound images in one or more data structures. For example, quality values may be associated with corresponding imaging parameter values in one data structure, or values for the quality metric may be associated with sets of ultrasound images in one data structure and the sets of ultrasound images may be associated with the corresponding imaging parameter values in another data structure. The processing circuitry may apply any maximum-finding algorithm to such a data structure/data structures in order to find the imaging parameter values that produced the set of ultrasound images having the highest quality. It should be appreciated that depending on the quality metric used, in some embodiments, lower values for the quality metric may be indicative of a higher quality for the ultrasound images (e.g., if the quality metric is a metric of how much noise is in the ultrasound images). In such embodiments, the processing circuitry may determine the set of ultrasound images having the lowest value for the quality value. In some embodiments, if multiple sets of parameters provide sets of ultrasound images having substantially the same quality, one of the sets of parameters may be chosen arbitrarily.

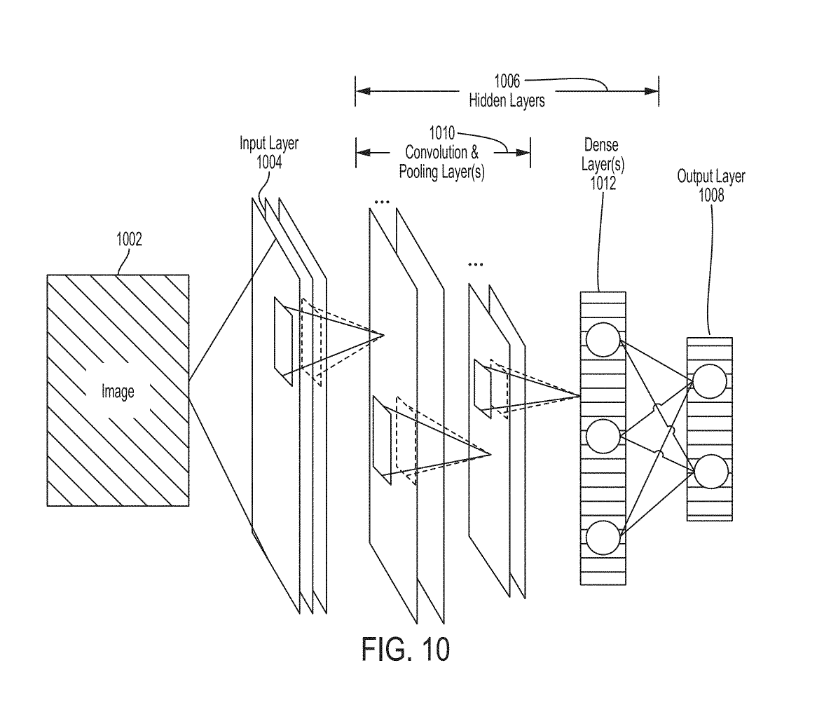

In some embodiments, determining the quality of a set of ultrasound images may include determining a confidence that a view classifier recognizes an anatomical region in the set of ultrasound images. In particular, the view classifier may include one or more convolutional neural networks trained to accept a set of ultrasound images (e.g., one or more ultrasound images) as an input and to recognize an anatomical region in the set of ultrasound images. Furthermore, the one or more convolutional neural networks may output a confidence (e.g., between 0% and 100%) in its classification of the anatomical region. The classification may include, for example, recognizing whether an anatomical region in an image represents an apical four chamber or apical two chamber view of the heart. To train the one or more convolutional neural networks to perform classification on images, the one or more convolutional neural networks may be trained with images that have been manually classified. For further description of convolutional neural networks and deep learning techniques, see the description with reference to FIG. 10. A high confidence that an anatomical region has been recognized may be indicative that the imaging parameter values used to produce the set of ultrasound images can be used to produce ultrasound images containing identifiable anatomical structures. Accordingly, a high confidence that an anatomical region has been recognized may correspond to a higher quality image.

In some embodiments, determining the quality of a set of ultrasound images may include determining an image sharpness metric. For example, determining the image sharpness metric for an ultrasound image may include calculating a two-dimensional Fourier transform of the ultrasound image, determining the centroid of the Fourier transformed image, and determining the maximum/minimum/mean/median/sum of the two frequencies at the centroid. A higher value for this metric may correspond to a higher quality image. Determining the image sharpness metric in this way may be more effective after a non-coherent compounding process configured to reduce speckle has been performed.

In some embodiments, determining the quality of a set of ultrasound images may include determining a pixel variation metric. For example, determining the pixel variation metric for an ultrasound image may include dividing an ultrasound image into blocks of pixels, finding the maximum pixel value within each block of pixels, determining the standard deviation of all the pixels in each block of pixels from the maximum pixel value within the block of pixels, and determining the maximum/minimum/mean/median/sum of all the standard deviations across all the blocks of pixels in the image. A lower value for this metric may correspond to a higher quality image.

In some embodiments, determining the quality of a set of ultrasound images may include determining a noise metric. For example, determining the noise metric for an ultrasound image may include using the CLEAN algorithm to find noise components within each pixel of the ultrasound image and determining the maximum/minimum/mean/median/sum of the noise components within each pixel of the ultrasound image. A lower value for this metric may correspond to a higher quality image.

In some embodiments, determining the quality of a set of ultrasound images may include determining a total variation metric for the image. For further description of the total variation metric, see Rudin, Leonid I., Stanley Osher, and Emad Fatemi. “Nonlinear total variation based noise removal algorithms.” Physica D: nonlinear phenomena 60.1-4 (1992): 259-268.

In some embodiments, determining the quality of a set of ultrasound images may include determining a pixel intensity metric. For example, determining the pixel intensity metric for an ultrasound image may include summing the absolute value/square/any power of the pixel intensities of the ultrasound image. A higher value for this metric may correspond to a higher quality image.

Further description of metrics for determining the quality of an image may be found in Kragh, Thomas J., and A. Alaa Kharbouch, “Monotonic iterative algorithm for minimum-entropy autofocus,” Adaptive Sensor Array Processing (ASAP) Workshop, (June 2006), Vol. 53, 2006; and Fienup, J. R., and J. J. Miller, “Aberration correction by maximizing generalized sharpness metrics,” JOSA A 20.4 (2003): 609-620, which are incorporated by reference herein in their entireties.

In some embodiments, one or more of the above metrics may be used in combination to determine the set of ultrasound images having the highest quality. For example, the sum/mean/median of two or more metrics may be used to determine the set of ultrasound images having the highest quality

In some embodiments, the processing circuitry may exclude portions of the set of ultrasound images that show reverberation or shadowing prior to determining the quality of a set of ultrasound images. A convolutional neural network may be trained to recognize reverberation or shadowing in portions of ultrasound images. In particular, the training data for the convolutional neural network may include portions of ultrasound images labeled with whether they exhibit reverberation, shadowing, or neither.

In act 112, the processing circuitry may automatically configure the ultrasound system to produce ultrasound images using a set of imaging parameter values with which the set of ultrasound images that has the highest quality was produced. Act 112 may be performed automatically by the processing circuitry after determining the set of ultrasound images that has the highest quality. For example, the processing device may transmit an instruction/instructions to the ultrasound imaging device to trigger configuration of the ultrasound imaging device with the imaging parameter values associated with the set of ultrasound images having the highest quality metric value determined in act 110. These imaging parameter values may be used by a user of the ultrasound system to continue imaging the region of interest.

In some embodiments, the process 100 may automatically proceed periodically. In other words, every time a set period of time elapses, the process 100 may automatically proceed in order to determine which set of imaging parameter values should be used for imaging during the next period of time. In other embodiments, the process 100 may automatically proceed based on the processing circuitry detecting that the ultrasound system has begun imaging the subject after not imaging the subject for a threshold period of time. In some embodiments, determining that the ultrasound system is not imaging a subject may include determining that the sum/mean/median of pixel values in a produced ultrasound image does not exceed a threshold value, and determining that the ultrasound system is imaging a subject may include determining that the sum/mean/median of pixel values in a produced ultrasound image does exceed a threshold value. In some embodiments, a convolutional neural network may be trained to recognize whether an ultrasound image was collected when an ultrasound imaging device was imaging a subject. The training data for the convolutional neural network may include ultrasound images labeled with whether the ultrasound image was collected when the ultrasound imaging device was imaging a subject or not. In some embodiments, determining whether the ultrasound system is imaging a subject may include calculating a cross-correlation between an ultrasound image collected by the ultrasound imaging device and a calibrated ultrasound image collected when there is an interface between an ultrasound imaging device and air. A cross-correlation having a mean to peak ratio that exceeds a threshold value (e.g., the peak cross-correlation value is over 20 times the mean cross-correlation value) may indicate that the ultrasound imaging device is not imaging a subject. In some embodiments, determining whether the ultrasound system is imaging a subject may include analyzing (e.g., using a fast Fourier transform) whether a period of intensities across an ultrasound image or across A-lines is highly correlated (e.g., the peak cross-correlation value is over 20 times the mean cross-correlation value), which may be indicative of reverberations and that there is an interface between the ultrasound imaging device and air. In some embodiments, determining whether the ultrasound system is imaging a subject may include calculating a cross-correlation over vertical components, such as columns of an image (or a subset of an image's columns and/or a subset of the pixels of columns of the image) collected perpendicular to the probe face or A-lines collected perpendicular to the probe face. If the ultrasound system is not imaging a subject, a mean to peak ratio of the cross-correlation may be over a specified threshold (e.g., the peak cross-correlation value may be over 20 times the mean cross-correlation value).

Detecting that the ultrasound system has begun imaging the subject after not imaging the subject for a threshold period of time may correspond to detecting the beginning of a new imaging session. Determining which set of imaging parameter values should be used for imaging at the beginning of an imaging session, but not during an imaging session, may be helpful for conserving power expended in producing multiple sets of ultrasound images and calculating values for a quality metric for each set of ultrasound images. Such embodiments may be appropriate in cases in which the region of a subject being scanned may not change in a way that would require substantial changes to imaging parameter values. For example, an imaging session including just imaging of the cardiac area may not require substantial changes to imaging parameter values during the imaging session. To conserve power while detecting whether the ultrasound system has begun imaging the subject after not imaging the subject for a threshold period of time, in some embodiments, detecting that the ultrasound system has begun imaging the subject may include configuring the ultrasound system with a set of imaging parameter values that use less power than the sets of imaging parameter values in act 104. (As referred to herein, a set of imaging parameter values that uses a certain amount or degree of power should be understood to mean that the ultrasound system uses the amount or degree of power when configured with the set of imaging parameter values). This may be a means of conserving power, as the ultrasound system may use lower power to collect ultrasound images of low but sufficient quality to detect that the ultrasound system has begun imaging a subject. Once this detection has occurred, the ultrasound system may use higher power to collect ultrasound images having higher quality sufficient for clinical use. The set of imaging parameter values that enables the ultrasound system to collect ultrasound image at lower power may include, for example, a lower pulse repetition frequency (PRF), lower frame rate, shorter receive interval, reduced number of transmits per image, and lower pulser voltage.

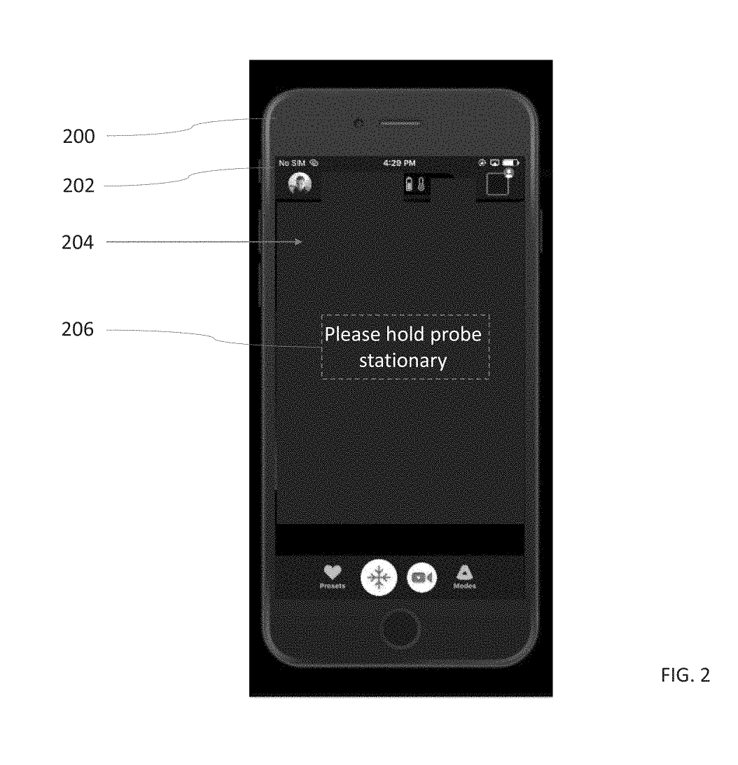

In some embodiments, until the processing circuitry has configured the ultrasound system to produce ultrasound images using the set of imaging parameter values with which the set of ultrasound images that has the highest quality was produced (i.e., until act 112 has been completed), the processing circuitry may generate a notification to hold the ultrasound imaging device substantially stationary (see, e.g., FIG. 2). This may be helpful in ensuring that all the imaging parameter values are evaluated for how appropriate they are for use at the particular region of interest. In some embodiments, the notification may be graphically displayed on a display of the processing device in operative communication with the ultrasound imaging device. In some embodiments, the notification may be played audibly by speakers of the processing device in operative communication with the ultrasound imaging device.

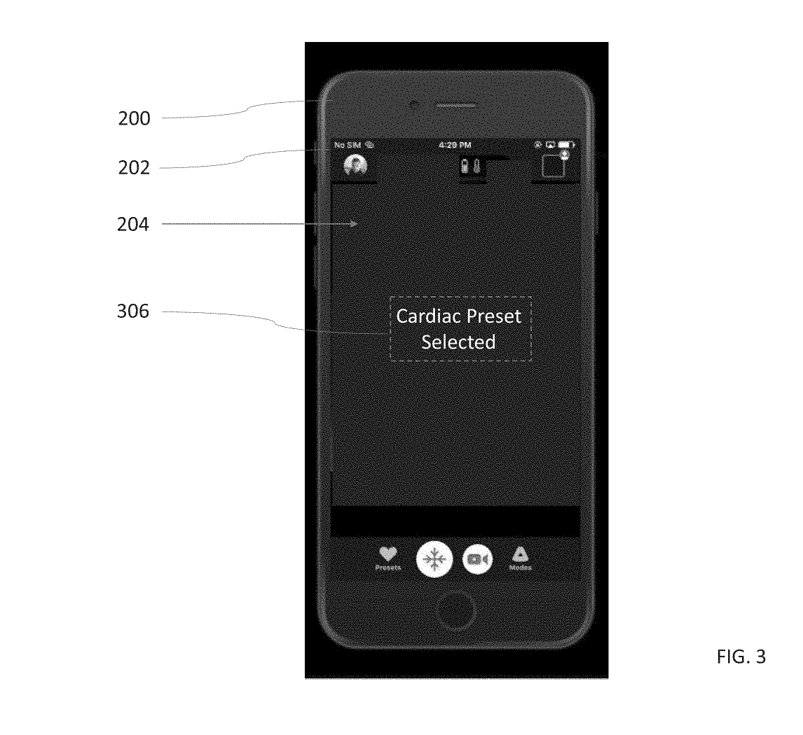

In some embodiments, once the processing circuitry has configured the ultrasound system to produce ultrasound images using the set of imaging parameter values with which the set of ultrasound images that has the highest quality was produced, the processing circuitry may generate a notification of which imaging parameter values were used to configure the ultrasound system for continued imaging at act 112 (see, e.g., FIGS. 3-6). For example, if a cardiac preset was used to configure the ultrasound system, the notification may indicate that a cardiac preset was used to configure the ultrasound system. In some embodiments, the notification may be graphically displayed on a display of the processing device in operative communication with the ultrasound imaging device. In some embodiments, the notification may be played audibly by speakers of the processing device in operative communication with the ultrasound imaging device. This may be helpful because the user may wish to use different imaging parameter values than the ones used to configure the ultrasound system at act 112. Through such a notification, the user may be able to determine if s/he needs to manually change the imaging parameter values used to configure the ultrasound system for continued imaging.

It should be appreciated that while the above description of process 100 references sets of ultrasound images (e.g., calculating the quality of sets of ultrasound images, inputting sets of ultrasound images to neural networks, etc.) the process 100 may also be applied to other types of ultrasound data (e.g., raw acoustical data, multilines, spatial coordinates, or other types of data generated from raw acoustical data).

FIG. 2 shows an example graphical user interface (GUI) 204 generated by a processing device 200 that may be in operative communication with an ultrasound imaging device, in which the GUI 204 shows a notification to hold the ultrasound imaging device stationary. As described above, it may be helpful to generate a notification to hold the ultrasound imaging device substantially stationary until an ultrasound system has been configured to produce ultrasound images using a set of imaging parameter values with which a set of ultrasound images that has the highest quality was produced. The processing device 200 includes a display 202 showing the GUI 204. The GUI 204 shows a graphical notification 206 to hold the ultrasound imaging device stationary. It should be appreciated that the exact form and text of the notification 206 is not limiting, and other forms and texts for the notification 206 that convey the similar intent may be used.

FIG. 3 shows an example GUI 304 generated by the processing device 200, in which the graphical user interface shows a textual notification of an automatically selected preset. As described above, it may be helpful to generate a notification of which imaging parameter values (e.g., preset) were used to configure an ultrasound system for continued imaging once the ultrasound system has been configured with the set of imaging parameter values that produced the highest quality ultrasound images. The processing device 200 includes the display 202 showing the GUI 304. The GUI 304 shows a textual notification 306 that a cardiac preset produced the highest quality set of images and has been selected for further imaging. It should be appreciated that while the example notification 306 indicates that a cardiac preset, the notification 306 may indicate that any preset or set of imaging parameter values has been selected. It should also be appreciated that the exact form and text of the notification 306 is not limiting, and other forms and texts for the notification 306 may be used.

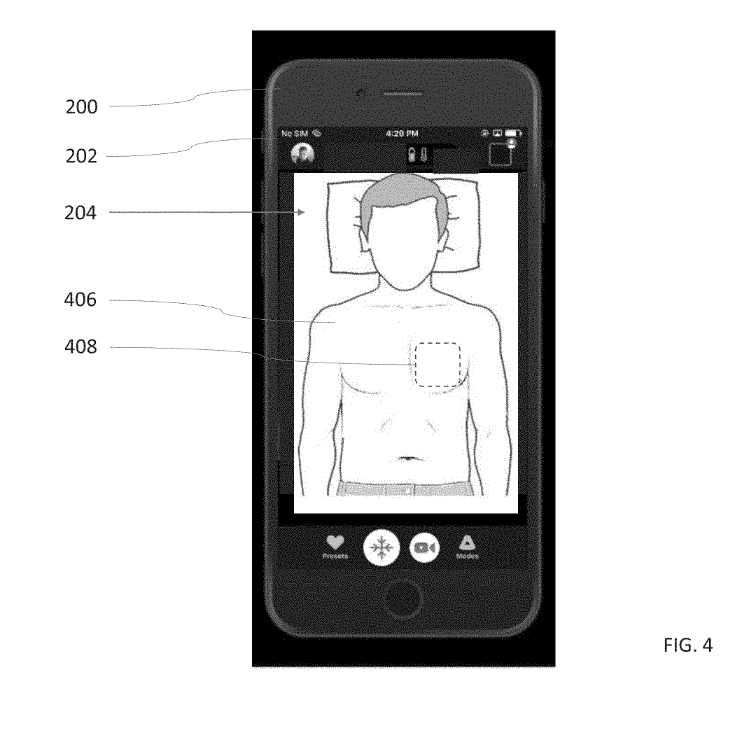

FIGS. 4-6 show example graphical user interfaces that may be useful, for example, in imaging protocols (e.g., FAST and RUSH) that include imaging multiple anatomic regions and may benefit from efficient automatic selection and changing of optimal imaging parameters depending on the anatomic region currently being imaged. FIG. 4 shows an example GUI 404 generated by the processing device 200, in which the GUI 404 shows a pictorial notification of an automatically selected preset. As described above, it may be helpful to generate a notification of which imaging parameter values (e.g., preset) were used to configure an ultrasound system for continued imaging once the ultrasound system has been configured with the set of imaging parameter values that produced the highest quality ultrasound images. The processing device 200 includes the display 202 showing the GUI 404. The GUI 404 shows an image of a subject 406 and an indicator 408. The indicator 408 indicates on the image of the subject 406 an anatomical region corresponding to the preset that produced the highest quality set of images and has been selected for further imaging. In the example of FIG. 4, the indicator 408 indicates that a cardiac preset has been chosen. It should be appreciated that while the example indicator 408 indicates the cardiac region, the indicator 408 may indicate any anatomical region. It should also be appreciated that the exact forms of the image of the subject 406 and the indicator 408 are not limiting, and other forms of the image of the subject 406 and the indicator 408 may be used. In some embodiments, the user may optionally change the preset selected by, for example, tapping another anatomical region on the image of the subject 406 on the GUI 404.

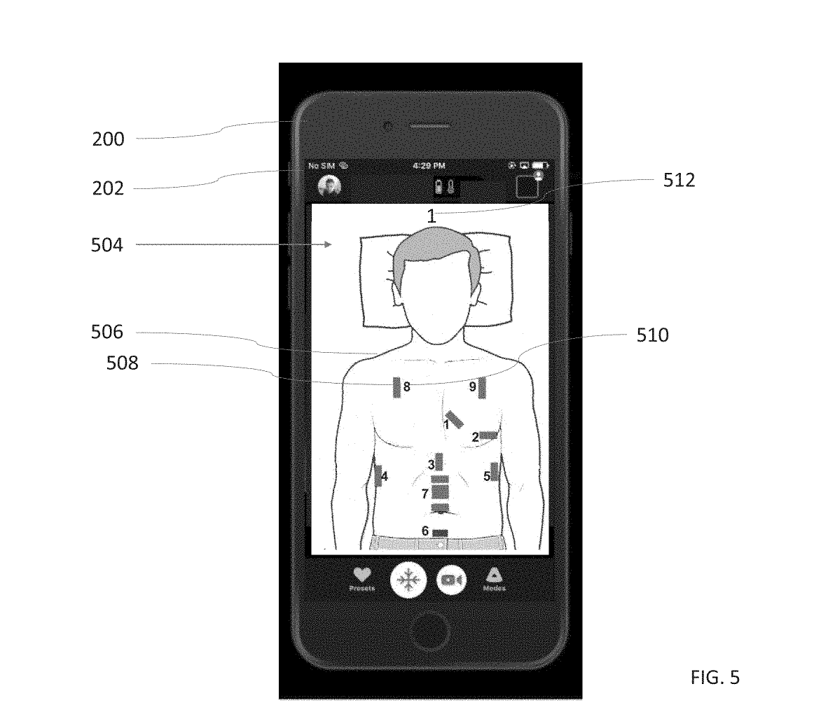

FIG. 5 shows a non-limiting alternative to the pictorial notification of FIG. 4. While FIG. 4 indicates the selected preset with the indicator 408, FIG. 5 indicates the selected preset on a GUI 504 with a number 512. The GUI 504 shows an image of a subject 506 and indications 508 of anatomical regions that are scanned as part of an imaging protocol. In the example of FIG. 5, the GUI 504 shows nine regions that are scanned as part of the RUSH protocol. The GUI 504 further shows numbers 510, each corresponding to one of the anatomical regions that is scanned as part of the imaging protocol. Additionally, the GUI 504 shows the number 512 at the top of the GUI 504. The number 512 matches one of the numbers 510 and thereby indicates which of the anatomical regions corresponds to the preset that produced the highest quality set of images and has been selected for further imaging. It should be appreciated that while the number 512 in FIG. 5 indicates the cardiac region, the number 512 may indicate any anatomical region. Additionally, while the indications 508 of anatomical regions corresponds to anatomical regions that may be scanned as part of the RUSH protocol, the indications 508 of anatomical regions may correspond to other imaging protocols. It should also be appreciated that the exact forms of the image of the subject 506, the indications 508 of anatomical regions, the numbers 510, and the number 512 are not limiting, and other forms of the image of the subject 506, the indications 508 of anatomical regions, the numbers 510, and the number 512. For example, the number 512 may be displayed in another region of the GUI 504. In some embodiments, if the user wishes to change the preset selected, the user may tap another anatomical region, indication 508, and/or number 510 on the GUI 504.

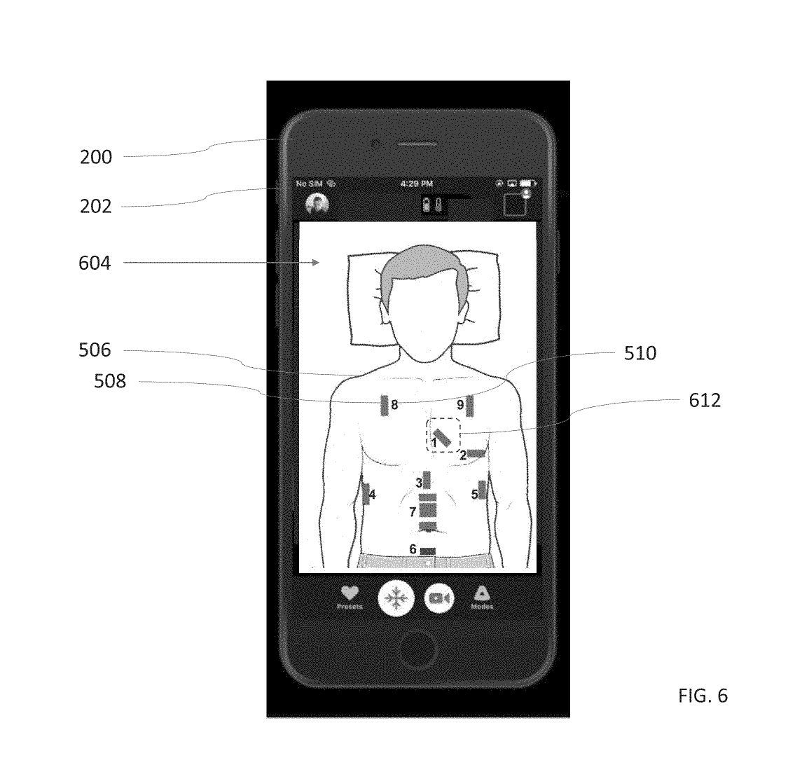

FIG. 6 shows another non-limiting alternative to the pictorial notifications of FIGS. 4 and 5. While FIGS. 4 and 5 indicate the selected preset with the indicator 408 and the number 512, respectively, FIG. 6 indicates the selected preset on the GUI 604 with an indicator 612. The indicator 612 highlights the anatomical region that corresponds to the preset that produced the highest quality set of images and has been selected for further imaging. In the example of FIG. 6, the indicator 612 encircles one of the indications 508 and one of the numbers 510. It should be appreciated that other manners for highlighting an anatomical region are possible, such as changing the color of the indication 508 and/or the number 510.

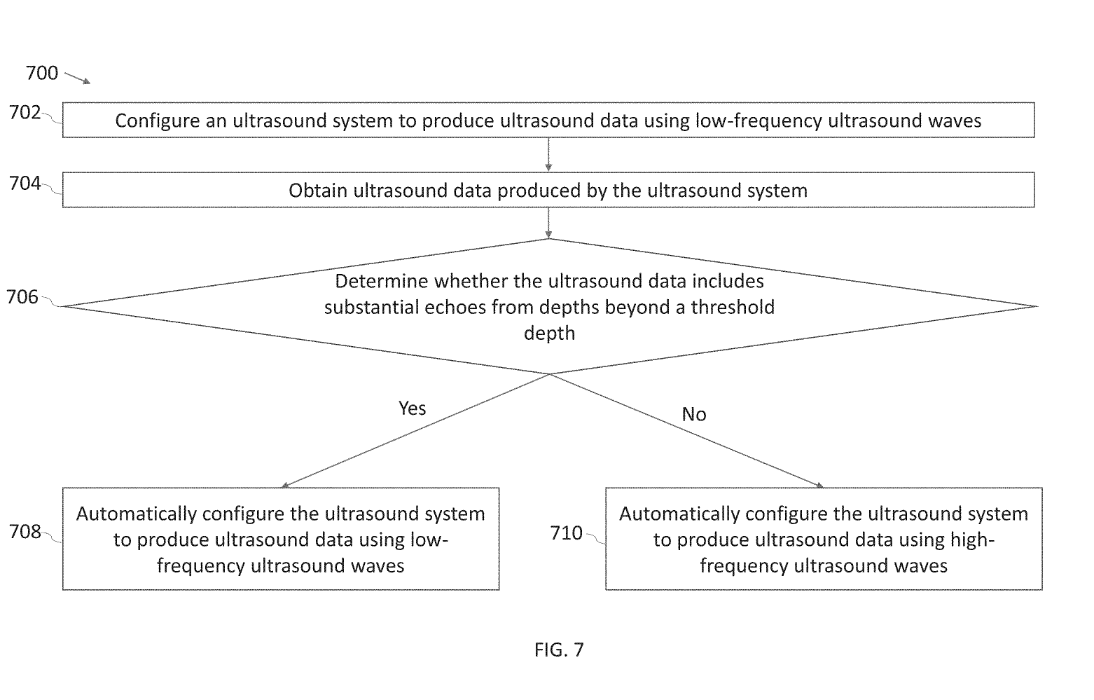

FIG. 7 shows an example process 700 for configuring an ultrasound system with imaging parameter values in accordance with certain embodiments described herein. The process 700 may be performed by, for example, processing circuitry in the ultrasound system. The ultrasound system may include an ultrasound imaging device used for imaging a subject as well as one or more external devices (e.g., a mobile phone, tablet, laptop, or server) in operative communication with the ultrasound imaging device, and the processing circuitry may be in either or both of these devices. Ultrasound systems and devices are described in more detail with reference to FIGS. 8-9.

Certain anatomical structures are located shallow within human subjects (e.g., 4-10 cm below the skin) and certain anatomical structures are located deep within human subjects (e.g., 10-25 cm below the skin). High-frequency ultrasound waves may be used to produce ultrasound images having higher axial resolution than images produced using low-frequency ultrasound waves. However, high-frequency ultrasound waves may be attenuated within a subject over a given distance than low-frequency ultrasound waves. Therefore, high-frequency ultrasound waves may be appropriate for ultrasound imaging of shallow anatomical structures, and low-frequency ultrasound may be appropriate for ultrasound imaging of deep anatomical structures. In the process 700, the processing circuitry may use a single test, namely production of ultrasound data from a region of interest on a subject using low-frequency ultrasound waves, to determine whether low-frequency ultrasound waves or high-frequency waves are appropriate for use in imaging the region of interest. To determine whether low-frequency ultrasound waves are appropriate for use in imaging the region of interest, the processing circuitry may determine whether substantial echoes are reflected back from beyond a threshold depth following transmission of test low-frequency ultrasound waves. If substantial echoes are reflected back from beyond the threshold depth following transmission of the test low-frequency ultrasound waves, this may indicate that deep anatomical structures are present and low-frequency ultrasound waves are appropriate for use. If substantial echoes are not reflected back from beyond the threshold depth following transmission of the test low-frequency ultrasound waves, this may indicate that deep anatomical structures are not present and high-frequency ultrasound waves are appropriate for use. The process 700 may be considered a method for automatically configuring an ultrasound system for deep or shallow ultrasound imaging.

In act 702, the processing circuitry may configure the ultrasound system to produce ultrasound data using low-frequency ultrasound waves. In some embodiments, a processing device in operative communication with the ultrasound imaging device may transmit an instruction/instructions to the ultrasound imaging device to trigger configuration of the ultrasound imaging device to produce ultrasound data using low-frequency ultrasound waves. In some embodiments, the low-frequency ultrasound waves may be in the range of approximately 1-5 MHz. The process 700 may then proceed to act 704.

In act 704, the processing circuitry may receive ultrasound data produced by the ultrasound system. The ultrasound data may be, for example, raw acoustical data, scan lines generated from raw acoustical data, and/or one or more ultrasound images generated from raw acoustical data. In some embodiments, after an ultrasound imaging device has received ultrasound data/images, the ultrasound imaging device may transmit the ultrasound data/images to a processing device in operative communication with the ultrasound imaging device. Transmission may occur over a wired communication link (e.g., over Ethernet, a Universal Serial Bus (USB) cable or a Lightning cable) or over a wireless communication link (e.g., over a BLUETOOTH, WiFi, or ZIGBEE wireless communication link). The process 100 may then proceed to act 706.

In act 706, the processing circuitry may determine whether the ultrasound data includes substantial echoes from depths beyond a threshold depth. For example, to determine whether raw acoustical data includes substantial echoes beyond a threshold depth, the processing circuitry may determine whether an amplitude of ultrasound waves received by the ultrasound imaging device exceeds a threshold amplitude value. In this example, the amplitude examined may be the amplitude of ultrasound waves received at the ultrasound imaging device after the time it takes for ultrasound waves to travel from the ultrasound imaging device to the threshold depth and reflect back from the threshold depth to the ultrasound imaging device. In particular, the time after which the amplitude of reflected ultrasound waves may be examined is approximately (2× threshold depth)/(speed of sound in tissue). The threshold depth may be, for example, a depth in the range of approximately 5-20 cm (e.g., 10-20 cm or 5-15 cm). To determine whether the amplitude of the ultrasound waves received by the ultrasound imaging device exceeds the threshold amplitude value, the processing circuitry may determine whether a peak amplitude and/or a mean amplitude of the ultrasound waves exceeds the threshold value.

As another example, a convolutional neural network accessed by the processing circuitry may be trained on raw acoustical data, scan lines generated from raw acoustical data, and/or ultrasound images generated from raw acoustical data, where the training data is manually labeled with whether the data includes substantial echoes from depths beyond a threshold depth. Using this training data, the convolutional neural network may be trained to determine whether inputted ultrasound data includes substantial echoes from depths beyond a threshold depth. If the processing circuitry determines, using the convolutional neural network, that the ultrasound data includes substantial echoes, the process 700 may proceed to act 708. If the processing circuitry determines, using the convolutional neural network, that the ultrasound data does not include substantial echoes from depths beyond a threshold depth, the process 700 may proceed to act 710.

In act 708, the processing circuitry may automatically configure the ultrasound system to produce ultrasound data using low-frequency ultrasound waves. Act 708 may be performed automatically by the processing circuitry after determining in act 706 that the ultrasound data produced in act 704 includes substantial echoes. For example, the processing device may transmit an instruction/instructions to the ultrasound imaging device to trigger configuration of the ultrasound imaging device to produce ultrasound data using low-frequency ultrasound waves. In some embodiments, the low-frequency ultrasound waves may be in the range of approximately 1-5 MHz.

In act 710, the processing circuitry may automatically configure the ultrasound system to produce ultrasound data using high-frequency ultrasound waves. Act 710 may be performed automatically by the processing circuitry after determining in act 706 that the ultrasound data produced in act 704 does not include substantial echoes. For example, the processing device may transmit an instruction/instructions to the ultrasound imaging device to trigger configuration of the ultrasound imaging device to produce ultrasound data using high-frequency ultrasound waves. In some embodiments, the high-frequency ultrasound waves may be in the range of approximately 5-15 MHz (e.g., 5-12 MHz or 8-15 MHz).

In some embodiments, the process 700 may automatically proceed periodically. In other words, every time a set period of time elapses, the process 700 may automatically proceed in order to determine whether low-frequency or high-frequency waves should be used. In other embodiments, the process 700 may automatically proceed based on the processing circuitry detecting that the ultrasound system has begun imaging the subject after not imaging the subject for a threshold period of time. Determining that the ultrasound system is not imaging a subject may include determining that the sum/mean/median of pixel values in a produced ultrasound image does not exceed a threshold value. Determining that the ultrasound system is imaging a subject may include determining that the sum/mean/median of pixel values in a produced ultrasound image does exceed a threshold value. Detecting that the ultrasound system has begun imaging the subject after not imaging the subject for a threshold period of time may correspond to detecting the beginning of a new imaging session. Determining whether low-frequency or high-frequency waves should be used for imaging at the beginning of an imaging session, but not during an imaging session, may be helpful for conserving power expended in determining whether collected ultrasound data includes substantial echoes from beyond the threshold depth. Such embodiments may be appropriate in cases in which the region of a subject being scanned may not change in a way that would require substantial changes to the frequency of ultrasound waves used. For example, an imaging session including just imaging of the cardiac area may not require substantial changes to ultrasound wave frequency during the imaging session.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

FIG. 8 shows a schematic block diagram illustrating aspects of an example ultrasound system 800 upon which various aspects of the technology described herein may be practiced. For example, one or more components of the ultrasound system 800 may perform any of the processes described herein. As shown, the ultrasound system 800 includes processing circuitry 801, input/output devices 803, ultrasound circuitry 805, and memory circuitry 807.

The ultrasound circuitry 805 may be configured to generate ultrasound data that may be employed to generate an ultrasound image. The ultrasound circuitry 805 may include one or more ultrasonic transducers monolithically integrated onto a single semiconductor die. The ultrasonic transducers may include, for example, one or more capacitive micromachined ultrasonic transducers (CMUTs), one or more CMOS ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 805 (e.g., transmit circuitry, receive circuitry, control circuitry, power management circuitry, and processing circuitry) to form a monolithic ultrasound imaging device.

The processing circuitry 801 may be configured to perform any of the functionality described herein. The processing circuitry 801 may include one or more processors (e.g., computer hardware processors). To perform one or more functions, the processing circuitry 801 may execute one or more processor-executable instructions stored in the memory circuitry 807. The memory circuitry 807 may be used for storing programs and data during operation of the ultrasound system 800. The memory circuitry 807 may include one or more storage devices such as non-transitory computer-readable storage media. The processing circuitry 801 may control writing data to and reading data from the memory circuitry 807 in any suitable manner.

In some embodiments, the processing circuitry 801 may include specially-programmed and/or special-purpose hardware such as an application-specific integrated circuit (ASIC). For example, the processing circuitry 801 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network.

The input/output (I/O) devices 803 may be configured to facilitate communication with other systems and/or an operator. Example I/O devices 803 that may facilitate communication with an operator include: a keyboard, a mouse, a trackball, a microphone, a touch screen, a printing device, a display screen, a speaker, and a vibration device. Example I/O devices 803 that may facilitate communication with other systems include wired and/or wireless communication circuitry such as BLUETOOTH, ZIGBEE, Ethernet, WiFi, and/or USB communication circuitry.

It should be appreciated that the ultrasound system 800 may be implemented using any number of devices. For example, the components of the ultrasound system 800 may be integrated into a single device. In another example, the ultrasound circuitry 805 may be integrated into an ultrasound imaging device that is communicatively coupled with a processing device that includes the processing circuitry 801, the input/output devices 803, and the memory circuitry 807.

FIG. 9 is a schematic block diagram illustrating aspects of another example ultrasound system 900 upon which various aspects of the technology described herein may be practiced. For example, one or more components of the ultrasound system 900 may perform any of the processes described herein. As shown, the ultrasound system 900 includes an ultrasound imaging device 914 in wired and/or wireless communication with a processing device 902. The processing device 902 includes an audio output device 904, an imaging device 906, a display screen 908, a processor 910, a memory 912, and a vibration device 909. The processing device 902 may communicate with one or more external devices over a network 916. For example, the processing device 902 may communicate with one or more workstations 920, servers 918, and/or databases 922.

The ultrasound imaging device 914 may be configured to generate ultrasound data that may be employed to generate an ultrasound image. The ultrasound imaging device 914 may be constructed in any of a variety of ways. In some embodiments, the ultrasound imaging device 914 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data.

The processing device 902 may be configured to process the ultrasound data from the ultrasound imaging device 914 to generate ultrasound images for display on the display screen 908. The processing may be performed by, for example, the processor 910. The processor 910 may also be adapted to control the acquisition of ultrasound data with the ultrasound imaging device 914. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

Additionally (or alternatively), the processing device 902 may be configured to perform any of the processes described herein (e.g., using the processor 910). For example, the processing device 902 may be configured to automatically determine an anatomical feature being imaged and automatically select, based on the anatomical feature being imaged, an ultrasound imaging preset corresponding to the anatomical feature. As shown, the processing device 902 may include one or more elements that may be used during the performance of such processes. For example, the processing device 902 may include one or more processors 910 (e.g., computer hardware processors) and one or more articles of manufacture that include non-transitory computer-readable storage media such as the memory 912. The processor 910 may control writing data to and reading data from the memory 912 in any suitable manner. To perform any of the functionality described herein, the processor 910 may execute one or more processor-executable instructions stored in one or more non-transitory computer-readable storage media (e.g., the memory 912), which may serve as non-transitory computer-readable storage media storing processor-executable instructions for execution by the processor 910.

In some embodiments, the processing device 902 may include one or more input and/or output devices such as the audio output device 904, the imaging device 906, the display screen 908, and the vibration device 909. The audio output device 904 may be a device that is configured to emit audible sound such as a speaker. The imaging device 906 may be configured to detect light (e.g., visible light) to form an image such as a camera. The display screen 908 may be configured to display images and/or videos such as a liquid crystal display (LCD), a plasma display, and/or an organic light emitting diode (OLED) display. The vibration device 909 may be configured to vibrate one or more components of the processing device 902 to provide tactile feedback. These input and/or output devices may be communicatively coupled to the processor 910 and/or under the control of the processor 910. The processor 910 may control these devices in accordance with a process being executed by the process 910 (such as the processes shown in FIGS. 1 and 7). Similarly, the processor 910 may control the audio output device 904 to issue audible instructions and/or control the vibration device 909 to change an intensity of tactile feedback (e.g., vibration) to issue tactile instructions. Additionally (or alternatively), the processor 910 may control the imaging device 906 to capture non-acoustic images of the ultrasound imaging device 914 being used on a subject to provide an operator of the ultrasound imaging device 914 an augmented reality interface.

It should be appreciated that the processing device 902 may be implemented in any of a variety of ways. For example, the processing device 902 may be implemented as a handheld device such as a mobile smartphone or a tablet. Thereby, an operator of the ultrasound imaging device 914 may be able to operate the ultrasound imaging device 914 with one hand and hold the processing device 902 with another hand. In other examples, the processing device 902 may be implemented as a portable device that is not a handheld device such as a laptop. In yet other examples, the processing device 902 may be implemented as a stationary device such as a desktop computer.

In some embodiments, the processing device 902 may communicate with one or more external devices via the network 916. The processing device 902 may be connected to the network 916 over a wired connection (e.g., via an Ethernet cable) and/or a wireless connection (e.g., over a WiFi network). As shown in FIG. 9, these external devices may include servers 918, workstations 920, and/or databases 922. The processing device 902 may communicate with these devices to, for example, off-load computationally intensive tasks. For example, the processing device 902 may send an ultrasound image over the network 916 to the server 918 for analysis (e.g., to identify an anatomical feature in the ultrasound) and receive the results of the analysis from the server 918. Additionally (or alternatively), the processing device 902 may communicate with these devices to access information that is not available locally and/or update a central information repository. For example, the processing device 902 may access the medical records of a subject being imaged with the ultrasound imaging device 914 from a file stored in the database 922. In this example, the processing device 902 may also provide one or more captured ultrasound images of the subject to the database 922 to add to the medical record of the subject. For further description of ultrasound imaging devices and systems, see U.S. patent application Ser. No. 15/415,434 titled “UNIVERSAL ULTRASOUND IMAGING DEVICE AND RELATED APPARATUS AND METHODS,” filed on Jan. 25, 2017 (and assigned to the assignee of the instant application), which is incorporated by reference herein in its entirety.

Aspects of the technology described herein relate to the application of automated image processing techniques to analyze images, such as ultrasound images. In some embodiments, the automated image processing techniques may include machine learning techniques such as deep learning techniques. Machine learning techniques may include techniques that seek to identify patterns in a set of data points and use the identified patterns to make predictions for new data points. These machine learning techniques may involve training (and/or building) a model using a training data set to make such predictions. The trained model may be used as, for example, a classifier that is configured to receive a data point as an input and provide an indication of a class to which the data point likely belongs as an output.

Deep learning techniques may include those machine learning techniques that employ neural networks to make predictions. Neural networks typically include a collection of neural units (referred to as neurons) that each may be configured to receive one or more inputs and provide an output that is a function of the input. For example, the neuron may sum the inputs and apply a transfer function (sometimes referred to as an “activation function”) to the summed inputs to generate the output. The neuron may apply a weight to each input, for example, to weight some inputs higher than others. Example transfer functions that may be employed include step functions, piecewise linear functions, and sigmoid functions. These neurons may be organized into a plurality of sequential layers that each include one or more neurons. The plurality of sequential layers may include an input layer that receives the input data for the neural network, an output layer that provides the output data for the neural network, and one or more hidden layers connected between the input and output layers. Each neuron in a hidden layer may receive inputs from one or more neurons in a previous layer (such as the input layer) and provide an output to one or more neurons in a subsequent layer (such as an output layer).

A neural network may be trained using, for example, labeled training data. The labeled training data may include a set of example inputs and an answer associated with each input. For example, the training data may include a plurality of ultrasound images or sets of raw acoustical data that are each labeled with an anatomical feature that is contained in the respective ultrasound image or set of raw acoustical data. In this example, the ultrasound images may be provided to the neural network to obtain outputs that may be compared with the labels associated with each of the ultrasound images. One or more characteristics of the neural network (such as the interconnections between neurons (referred to as edges) in different layers and/or the weights associated with the edges) may be adjusted until the neural network correctly classifies most (or all) of the input images.

Once the training data has been created, the training data may be loaded to a database (e.g., an image database) and used to train a neural network using deep learning techniques. Once the neural network has been trained, the trained neural network may be deployed to one or more processing devices. It should be appreciated that the neural network may be trained with any number of sample patient images. For example, a neural network may be trained with as few as 7 or so sample patient images, although it will be appreciated that the more sample images used, the more robust the trained model data may be.

In some applications, a neural network may be implemented using one or more convolution layers to form a convolutional neural network. An example convolutional neural network is shown in FIG. 10 that is configured to analyze an image 1002. As shown, the convolutional neural network includes an input layer 1004 to receive the image 1002, an output layer 1008 to provide the output, and a plurality of hidden layers 1006 connected between the input layer 1004 and the output layer 1008. The plurality of hidden layers 1006 includes convolution and pooling layers 1010 and dense layers 1012.

The input layer 1004 may receive the input to the convolutional neural network. As shown in FIG. 10, the input the convolutional neural network may be the image 1002. The image 1002 may be, for example, an ultrasound image.

The input layer 1004 may be followed by one or more convolution and pooling layers 1010. A convolutional layer may include a set of filters that are spatially smaller (e.g., have a smaller width and/or height) than the input to the convolutional layer (e.g., the image 1002). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 1010 may be followed by dense layers 1012. The dense layers 1012 may include one or more layers each with one or more neurons that receives an input from a previous layer (e.g., a convolutional or pooling layer) and provides an output to a subsequent layer (e.g., the output layer 1008). The dense layers 1012 may be described as “dense” because each of the neurons in a given layer may receive an input from each neuron in a previous layer and provide an output to each neuron in a subsequent layer. The dense layers 1012 may be followed by an output layer 1008 that provides the output of the convolutional neural network. The output may be, for example, an indication of which class, from a set of classes, the image 1002 (or any portion of the image 1002) belongs to.

It should be appreciated that the convolutional neural network shown in FIG. 10 is only one example implementation and that other implementations may be employed. For example, one or more layers may be added to or removed from the convolutional neural network shown in FIG. 10. Additional example layers that may be added to the convolutional neural network include: a rectified linear units (ReLU) layer, a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output.

For further description of deep learning techniques, see U.S. patent application Ser. No. 15/626,423 titled “AUTOMATIC IMAGE ACQUISITION FOR ASSISTING A USER TO OPERATE AN ULTRASOUND IMAGING DEVICE,” filed on Jun. 19, 2017 (and assigned to the assignee of the instant application), which is incorporated by reference herein in its entirety. In any of the embodiments described herein, instead of/in addition to using a convolutional neural network, a fully connected neural network may be used.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An ultrasound system, comprising:
a processing device configured to::
automatically image an anatomical target multiple times with different sets of imaging parameters; and
automatically select for continued imaging of the anatomical target, from the different sets of imaging parameters, a first set of imaging parameters.

2. The ultrasound system of claim 1, wherein the first set of imaging parameters represents those imaging parameters determined to produce images of the anatomical target of a highest quality from among the sets of imaging parameters

3. An ultrasound system, comprising:
a processing device configured to:
configure the ultrasound system to produce a plurality of sets of ultrasound images, each respective set of the plurality of sets of ultrasound images being produced with a different respective set of a plurality of sets of imaging parameter values;
obtain, from the ultrasound system, the plurality of sets of ultrasound images;
determine a set of ultrasound images from among the plurality of sets of ultrasound images that has a highest quality; and
based on determining the set of ultrasound images from among the plurality of sets of ultrasound images that has the highest quality, automatically configure the ultrasound system to produce ultrasound images using a set of imaging parameter values with which the set of ultrasound images that has the highest quality was produced.

4. The ultrasound system of claim 3, wherein the processing device is configured to configure the ultrasound system to produce the plurality of sets of ultrasound images based on detecting that the ultrasound system has begun imaging a subject after not imaging the subject for a threshold period of time.

5. The ultrasound system of claim 4, wherein the processing device is configured, when detecting that the ultrasound system has begun imaging the subject after not imaging the subject for the threshold period of time, to configure the ultrasound system with a low-power set of imaging parameter values that uses less power than the plurality of sets of imaging parameter values.

6. The ultrasound system of claim 3, wherein the processing device is configured, when determining the set of ultrasound images from among the plurality of sets of ultrasound images that has the highest quality, to determine the set of ultrasound images from among the plurality of sets of ultrasound images for which a view classifier has a highest confidence that the view classifier recognizes an anatomical region in the set of ultrasound images. .

7. The ultrasound system of claim 3, wherein the processing device is configured, when determining the set of ultrasound images from among the plurality of sets of ultrasound images that has the highest quality, to calculate an image sharpness metric for each of the plurality of sets of ultrasound images.

8. The ultrasound system of claim 3, wherein the processing device is configured, when determining the set of ultrasound images from among the plurality of sets of ultrasound images that has the highest quality, to calculate a pixel variation metric for each of the plurality of sets of ultrasound images.

9. The ultrasound system of claim 3, wherein the processing device is configured, when determining the set of ultrasound images from among the plurality of sets of ultrasound images that has the highest quality, to calculate a noise metric for each of the plurality of sets of ultrasound images.

10. The ultrasound system of claim 3, wherein the processing device is configured, when determining the set of ultrasound images from among the plurality of sets of ultrasound images that has the highest quality, to calculate a total variation metric for each of the plurality of sets of ultrasound images.

11. The ultrasound system of claim 3, wherein the processing device is configured, when determining the set of ultrasound images from among the plurality of sets of ultrasound images that has the highest quality, to calculate a pixel intensity metric for each of the plurality of sets of ultrasound images.

12. The ultrasound system of claim 3, wherein the processing device is further configured to generate an instruction for a user to hold substantially stationary an ultrasound imaging device configured for operative communication with the processing device while the ultrasound system is producing the plurality of sets of ultrasound images.

13. The ultrasound system of claim 3, wherein the processing device is further configured to generate a notification for a user that indicates the set of imaging parameter values with which the set of ultrasound images that has the highest quality metric was produced.

14. The ultrasound system of claim 3, wherein the plurality of sets of imaging parameter values comprise ultrasound imaging presets each optimized for imaging a particular anatomical region among a plurality of anatomical regions.

15. The ultrasound system of claim 14, wherein the plurality of anatomical regions comprise a plurality of anatomical regions typically imaged during a particular ultrasound imaging protocol.

16. The ultrasound system of claim 15, wherein the processing device is further configured to receive an input from a user that the user will be performing the particular ultrasound imaging protocol.

17. The ultrasound system of claim 3, wherein the plurality of sets of imaging parameter values comprise preferred sets of imaging parameter values associated with a user.

18. The ultrasound system of claim 3, wherein the processing device is configured, when configuring the ultrasound system to produce the plurality of sets of ultrasound images, to:
configure the ultrasound system to:
transmit a plurality of sets of ultrasound waves into a subject using the plurality of sets of imaging parameter values, wherein the plurality of sets of imaging parameter values relate to ultrasound transmission; and
generate each of the plurality of sets of ultrasound images from a different set of reflected ultrasound waves each corresponding to one of the plurality of sets of transmitted ultrasound waves.

19. The ultrasound system of claim 3, wherein the processing device is configured, when configuring the ultrasound system to produce the plurality of sets of ultrasound images, to:
configure the ultrasound system to:
transmit a single set of ultrasound waves into a subject; and
generate each of the plurality of sets of ultrasound images from a single set of reflected ultrasound waves corresponding to the single set of transmitted ultrasound waves using the plurality of sets of imaging parameter values, wherein the plurality of sets of imaging parameter values relate to ultrasound image generation.

20. The ultrasound system of claim 3, wherein the ultrasound system includes the processing device and an ultrasound imaging device.

21. The ultrasound system of claim 3, wherein the ultrasound system includes the processing device.
